SEQUENCE LISTING (16 sequences)

SEQ ID NO: 1 - protein, 124 aa, Artificial Sequence
  V domain of H chain of humanized antibody hB-F5H37L
EEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSS

SEQ ID NO: 2 - protein, 111 aa, Artificial Sequence
  V domain of K chain of humanized antibody hB-F5L4M
DIVMTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIK

SEQ ID NO: 3 - DNA, 550 bp, Artificial Sequence
  Part of plasmid encoding V domain of H chain of humanized antibody hB-F5
gaggagctccagacaatgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcagaggaacagcttgtggagtctgggggaggcttggtgaaacccggaggttctctgaggctctcctgtgcagcctcgggtttcagtttcagtgactgccggatgtactgggttcgccaggctccagggaaggggctggagtggattggtgtgatttcagtcaaatctgagaattatggagcaaattatgcagagtctgtgaggggcagattcactatttcaagagatgattcaaaaaacacggtctatctgcagatgaacagcttgaagaccgaagacactgccgtttattattgtagtgcctcctattataggtacgacgtgggggcctggtttgcttactggggccaagggactctggtcactgtctcttcaggtaagaatggccaagcttg

SEQ ID NO: 4 - DNA, 498 bp, Artificial Sequence
  Part of plasmid encoding V domain of K chain of humanized antibody hB-F5
ggaggatccaattatctgctgacttataatactactagaaagcaaatttaaatgacatatttcaattatatctgagacagcgtgtataagtttatgtataatcattgtccattcctgactacaggtgcctacggggacatcgtgatgacccagtctccagactccctggctgtgtctctgggcgagagggccaccatcaactgcagggccagcaaaagtgtcagtacatctggctacagttatatatattggtaccagcagaaaccaggacagcctcctaagctgctcatttaccttgcatccatcctagaatctggggtccctgaccgattcagtggcagcgggtctgggacagatttcactctcaccatcagcagcctgcaggctgaagatgtggcagtttattactgtcagcacagtagggaacttccgtggacgttcggccaagggaccaaggtggaaatcaaacgtgagtagaatttaaattttaagcttctt

SEQ ID NO: 5 - protein, 124 aa, Mus musculus
QEYLVETGGGLVRPGNSLKLSCVTSGFSFSDCRMYWLRQPPGKGLEWIGVISVKSENYGANYAESVRGRFTISRDDSKSSVYLQMSRLREEDTATYYCSASYYRYDVGAWFAYWGQGTLVTVSA

SEQ ID NO: 6 - protein, 111 aa, Mus musculus
DIVLTQSPSSLVVSLGQRATISCRASKSVSTSGYSYIYWYQQIPGQPPKLLIYLASILESGVPGRFSGSGSGTDFTLNIHPVEEEDAATYYCQHSRELPWTFGGGTKLEIK

SEQ ID NO: 7 - protein, 23 aa, Homo sapiens
DIVMTQSPDSLAVSLGERATINC

SEQ ID NO: 8 - protein, 15 aa, Homo sapiens
WYQQKPGQPPKLLIY

SEQ ID NO: 9 - protein, 32 aa, Homo sapiens
GVPDRFSGSGSGTDFTLTISSLQAEDVAVYYC

SEQ ID NO: 10 - protein, 10 aa, Homo sapiens
FGQGTKVEIK

SEQ ID NO: 11 - protein, 30 aa, Homo sapiens
EVQLVESGGGLVKPGGSLRLSCAASGFTFS

SEQ ID NO: 12 - protein, 14 aa, Homo sapiens
WVRQAPGKGLEWVG

SEQ ID NO: 13 - protein, 32 aa, Homo sapiens
RFTISRDDSKNTLYLQMNSLKTEDTAVYYCTT

SEQ ID NO: 14 - protein, 11 aa, Homo sapiens
WGQGTLVTVSS

SEQ ID NO: 15 - protein, 124 aa, Artificial Sequence
  V domain of the H chain of humanized antibody hB-F5H37V
EEQLVESGGGLVKPGGSLRLSCAASGFSFSDCRMYWVRQAPGKGLEWIGVISVKSENYGANYAESVRGRFTISRDDSKNTVYLQMNSLKTEDTAVYYCSASYYRYDVGAWFAYWGQGTLVTVSS

SEQ ID NO: 16 - protein, 111 aa, Artificial Sequence
  V domain of K chain of humanized antibody hB-F5L4L
DIVLTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIK

(19) United States
(12) Patent Application Publication
(10) Pub. No.: US 2011/0229465 A1
(43) Pub. Date: Sep. 22, 2011

(54) COMPOSITION FOR TREATING DISEASE

(76) Inventors: Frank Osterroth, Dietzenbach (DE); Silke Aigner, Frankenthal (DE); Christoph Uherek, Seligenstadt (DE); Andrea Wartenberg-Demand, Linden (DE); Anatoly Rudnev, Bad Homburg (DE); Michael Soldan, Biedenkopf (DE); Christoph Bruecher, Eschborn (DE); Benjamin Daelken, Frankfurt am Main (DE); Chantal Zuber, Frankfurt am Main (DE); Gregor Schulz, Umkirch (DE); Niklas Czeloth, Dreieich (DE)

(21) Appl. No.: 13/074,357

(22) Filed: Mar. 29, 2011

Related U.S. Application Data
(63) Continuation of application No. PCT/EP2009/061210, filed on Aug. 31, 2009.

(30) Foreign Application Priority Data
Sep. 29, 2008 (GB) 0817809.7
Sep. 29, 2008 (GB) 0817810.5
Sep. 29, 2008 (GB) 0817811.3
Mar. 10, 2009 (EP) PCT/EP2009/052809
Mar. 10, 2009 (EP) PCT/EP2009/052810
Mar. 10, 2009 (EP) PCT/EP2009/052811

(51) Int. Cl. A61K 39/395; A61K 31/519; A61K 38/13; A61K 31/675; A61K 31/65; A61K 31/575; A61P 19/02; A61P 29/00

(52) U.S. Cl. 424/133.1; 514/249; 424/172.1; 514/20.5; 514/110; 514/154; 514/171

(57) ABSTRACT

The present invention provides pharmaceutical compositions and kits comprising an agent capable of activating CD4+CD25+ regulatory T cells and methotrexate, and methods of treatment and medical uses utilising the same.

This application is a Continuation of, and claims priority under 35 U.S.C. §120 to, International Application No. PCT/EP2009/061210, filed Aug. 31, 2009, and claims priority therethrough under 35 U.S.C. §119 and/or §365 to Great Britain Patent Application No. 0817810.5, filed Sep. 29, 2008, Great Britain Patent Application No. 0817811.3, filed Sep. 29, 2008, Great Britain Patent Application No. 0817809.7, filed Sep. 29, 2008, PCT Patent Application No. PCT/EP2009/052811, filed Mar. 10, 2009, PCT Patent Application No. PCT/EP2009/052809, filed Mar. 10, 2009, and PCT Patent Application No. PCT/EP2009/052810, filed Mar. 10, 2009, the entireties of which are incorporated by reference herein. Also, the Sequence Listing filed electronically herewith is hereby incorporated by reference (File name: 2011-03-29T_{—}060-012_Seq_List; File size: 11 KB; Date recorded: Mar. 29, 2011).

### BACKGROUND OF THE INVENTION

1. Field of the Invention

The present invention is concerned with treatment of rheumatic diseases. The invention involves a highly effective pharmaceutical composition comprising an agent capable of activating CD4+CD25+ regulatory T cells, such as a humanised monoclonal antibody, and the drug methotrexate. The composition and kits of the invention are particularly effective in the treatment of rheumatoid arthritis. The invention envisages pharmaceutical compositions or kits comprising the agent and methotrexate, as well as uses and methods of treatment employing the composition and kits.

2. Brief Description of the Related Art

Rheumatic diseases are a group of diseases affecting the connective tissue, especially the joints and related structures, and are characterized by inflammation, degeneration or metabolic derangement. Examples of rheumatic diseases are rheumatoid arthritis, psoriatic arthritis, juvenile rheumatoid arthritis and ankylosing spondylitis.

Rheumatoid arthritis is an autoimmune disease which causes chronic inflammation of joints and surrounding tissues, and can also affect other tissues and body organs. The disease occurs when T cells, which are normally tolerant with regard to autologous tissue, recognise and react to ‘self’ molecules, that is, molecules produced by the cells of the host. Activation of ‘autoreactive’ T cells by presentation of autoantigens processed by antigen presenting cells (APC) leads to their clonal expansion and migration to the specific tissues, where they induce inflammation and tissue destruction.

There are a large range of treatments for rheumatoid arthritis which are currently available, including first line drugs for controlling pain and inflammation classified as non-steroidal anti-inflammatory drugs (NSAIDs), e.g., aspirin, ibuprofen, naproxen, etc. Secondary treatment of arthritis includes corticosteroids (e.g. prednisone and dexamethasone), which are synthetic versions of the body's cortisone hormone, slow acting antirheumatic drugs (SAARDs) or disease-modifying anti-rheumatic drugs (DMARDs), e.g., hydroxycloroquine, sulfasalazine, methotrexate, penicillinamine, cyclophosphamide, gold salts, azothipoprine, leflunomide, etc.

Many patients with newly diagnosed RA are started with a DMARD, such as methotrexate (MTX). MTX (4-amino-N10-methylpteroyl glutamic acid) is an analogue of folic acid, which is known to hamper the production of a form of folic acid important for actively growing cells such as those found in the skin, blood, gastrointestinal tissues and those involved in the immune system. It is not entirely clear how MTX decreases the severity of RA, however, it is thought to play a role in anti-inflammatory action and a variety of pharmacological mechanisms for its action have been proposed, including inhibition of purine synthesis, promotion of adenosine release, inhibition of production of proinflammatory cytokines, and modulation of inflammation (Swierkot et al., 2006). MTX is also known to inhibit, for example, the activity of an enzyme called dihydrofolate reductase (DHFR), and also interferes with several other enzymes.

Another group of drugs for the treatment of RA are called biological-response modifiers (BRMs), which includes monoclonal antibodies. Examples of these are antagonists to tumour necrosis factor-alpha (TNF-alpha), such as adalimumab, infliximab, and etanercept, which work through binding to the TNF-alpha receptor or directly binding to the TNF-alpha protein itself. Several TNF-alpha inhibitors have been approved by the FDA for treatment of rheumatoid arthritis, including adalimumab (Humira®), Infliximab (Remicade®) and Etanercept (Enbrel®). TNF-alpha represents a key mediator in rheumatoid arthritis, and is mainly produced by activated macrophages within the synovium of RA patients. Acting as a pro-inflammatory cytokine, TNF alpha is abundantly present in the synovial tissue of RA patients. It induces the production and release of chemokines that attract leukocytes from the blood into the inflamed tissue (Tracey et al., 2008). Beside the mediation of synovial inflammation, TNF alpha is involved in joint destruction and cartilage degradation. Additionally, it is capable of inhibiting the suppressive activity of CD4+CD25+ regulatory T-cells (Andersson et al., 2008).

In some cases RA patients are treated with a combination of the drugs discussed above. In particular, the DMARDs are frequently used as a first treatment. However, it can be desirable in patients where disease control is not achieved, to use them in combination with treatments that have been more recently approved, such as biological agents e.g. TNF-alpha antagonists. It has been reported that a combination of MTX with some monoclonal antibodies (etanercept, infliximab, adalimumab and anakirina) leads to a better therapeutic efficacy as compared to MTX therapy alone (Swierkot et al., 2006). However, MTX exerts a variety of pharmacological actions and its clinical effects can be attributed to multiple targets (Wessels et al., 2008). Accordingly, it cannot readily be predicted how MTX will affect the therapeutic activity, and therefore the efficacy, of a drug which is effective as a single agent.

Despite the range of currently available drugs, not all patients respond well to the above treatments and there are a number of adverse side effects. For example, TNF-alpha treatment down regulates the immune system making the treated patients more susceptible to infections and disease. Accordingly, there is still a need for alternative therapies to be developed.

It is generally agreed that CD4⁺ T cells play a major part in initiating and maintaining autoimmunity. Accordingly, it has been proposed to use mAbs against CD4⁺ T cells surface molecules, and in particular anti-CD4 mAbs, as immunosuppressive agents in the treatment of diseases such as rheumatoid arthritis.

One example under further investigation is the anti-CD4 B-F5 antibody (murine IgG1 anti-human CD4), which has been tested in different autoimmune diseases. In rheumatoid arthritis patients, the results observed in a placebo controlled trial with a daily dose of B-F5 did not indicate a significant improvement (Wendling et al. J Rheumatol; 25(8):1457-61, 1998). However, in WO 2004/083247, a humanized B-F5 (hereinafter referred to as hB-F5 or BT061) antibody having similar CD4 binding properties to the parent mouse B-F5 was developed. A preliminary evaluation of the effect of the humanized version of the mouse B-F5 antibody in patients also receiving the non-steroidal anti-inflammatory drug Diclophenac provided an indication of an effective immunosuppression, reflected by a positive clinical effect in the patients when used in a 10 day treatment.

The study was also described by Wijdenes et al., in an abstract and poster presented at the EULAR conference, June 2005. They described the treatment of 11 patients suffering from rheumatoid arthritis with 5 intravenous infusions of 5 mg hB-F5 every other day with concomitant treatment with 150 mg Diclophenac (Wijdenes et al., Abstract and poster, EULAR conference, June 2005).

In WO 2004/083247 it was noted that the antibody was able to activate a particular subset of CD4+ T cells, namely CD4+CD25+ regulatory T cells (Tregs). These cells constitute 5-10% of peripheral CD4+ T cells and, once stimulated, are competent to suppress the response of CD4+ T cells and CD8+ T cells as well as inhibit B-cell activation and clonal expansion. Thus these cells represent an important level of control in the immune system. In particular, CD4⁺CD25⁺Treg cells are involved in maintaining immune homeostasis in the periphery, and regulating autoimmunity and pathogenic immune responses.

The treatment of rheumatoid arthritis via a mechanism which activates CD4+CD25+ regulatory T cells represents an important avenue of research, and the use of the hB-F5 agent in phase II clinical trials on patients suffering from rheumatoid arthritis is discussed in International Patent Application numbers PCT/EP2009/052809, PCT/EP2009/052811 and PCT/EP2009/052810.

However, it cannot readily be predicted whether any new treatment can be successfully combined with the current treatments to give a beneficial therapeutic effect. As mentioned above, this is particularly the case with MTX.

Several studies have reported findings which suggest that MTX has a negative effect on regulatory T cells and therefore is likely to prevent MTX being used in a combination therapy with an agent which relies on activation of CD4+CD25+ regulatory T cells for its therapeutic mechanism. Wascher et al., (1994) and Herman et al., (2005) report findings that suggest that MTX may reduce the number of available T lymphocytes. Wascher et al., reported that administration of high dose MTX given intravenously at 12 weeks significantly (P<0.01) reduced total peripheral blood lymphocytes and led to a pronounced redistribution of lymphocyte subsets with a preferred reductive effect on B-lymphocytes (P<0.005) and T lymphocytes (P<0.05). Herman et al., 2005 reported an apoptosis inducing effect of MTX in T lymphocytes in vitro, at concentrations reflecting a low dose therapy of RA patients (7.5 mg).

Further, an in vitro study conducted by Porter et al., 2006 reported an influence of MTX on the viability of regulatory T-cells. At in vitro concentrations of 50 nM (highest concentration analyzed) the suppressive activity of Treg cells was significantly reduced from 94% to 88% (p<0.05). This suggests that the presence of MTX may inhibit Treg suppression.

Still further, Yamaguchi et al., 2007 reported that natural Treg cells constitutively expressed high amounts of folate receptor 4 (FR4). Since MTX is a folate analogue, it is suggested that MTX may also be taken up by Tregs cells. Such an uptake is likely to result in interference with metabolism within this cell population.

Moreover, it is known that the therapeutic activity of many antibodies is influenced by Fc receptors on Fc receptor expressing cells. Some antibodies even need to bind to Fc receptors on Fc receptor expressing cells to be active. However, MTX therapy is known to result in a decrease of Fc gamma R1 expression on monocytes (Wijngaarden et al., 2004, 2005) in vivo. The reductive influence of MTX on Fc receptor expression on monocytes has also been demonstrated in patients that were treated with MTX and the therapeutic anti-TNF-alpha antibody Infliximab (Wijngaarden et al., 2008). As a result, it is generally considered that MTX negatively influences the activity of Fc receptor binding antibodies. Accordingly, it is expected that MTX will negatively influence the capacity of an antibody to activate Tregs.

Given the above, it is expected that MTX will have a negative impact on the therapeutic capacity of an agent which works via the activation of CD4+CD25+ regulatory T cells, such as hB-F5. As such, it can be seen that the outcome of the combinative treatment approaches cannot be predicted.

### SUMMARY OF THE INVENTION

Having regard to the above described prior art, it is the aim of the present invention to develop further and improved pharmaceutical compositions for the treatment of rheumatoid arthritis.

Accordingly, the present invention provides a pharmaceutical composition comprising an agent capable of activating CD4+CD25+ regulatory T cells and methotrexate. The present invention further provides a kit comprising separately an agent capable of activating CD4+CD25+ regulatory T cells and methotrexate.

The present inventors have unexpectedly found that a combination of an agent capable of activating CD4+CD25+ regulatory T cells with methotrexate has a therapeutic effect, and is surprisingly advantageous in relation to the reduced number of antibody-related side effects. The combination is also surprisingly advantageous in relation to the speed at which a high level therapeutic effect is reached.

Accordingly, the present invention also provides a method of treating a rheumatic disease in a patient comprising a step (a) of administering an agent capable of activating CD4+CD25+ regulatory T cells and a step (b) of administering methotrexate, wherein step (a) and step (b) can be conducted simultaneously, separately or sequentially and in either order.

Further, the present invention provides a method of treating a rheumatic disease in a patient undergoing methotrexate treatment comprising a step of administering an agent capable of activating CD4+CD25+ regulatory T cells. Alternatively, the present invention provides a method of treating a rheumatic disease in a patient undergoing treatment with an agent capable of activating CD4+CD25+ regulatory T cells comprising a step of administering methotrexate.

Still further, the present invention provides a method of treating rheumatoid arthritis in a patient who is a non-responder to treatment with a disease-modifying anti-rheumatic drug (DMARD), comprising a step (a) of administering an agent capable of activating CD4+CD25+ regulatory T cells and a step (b) of administering methotrexate, wherein step (a) and step (b) can be conducted simultaneously, separately or sequentially and in either order.

In addition the present invention provides an agent capable of activating CD4+CD25+ regulatory T cells and methotrexate as a combined preparation for simultaneous, separate or sequential use in medicine. In one aspect, the present invention provides an agent capable of activating CD4+CD25+ regulatory T cells and methotrexate as a combined preparation for simultaneous, separate or sequential use in the treatment of a rheumatic disease. In an alternative aspect the present invention provides an agent capable of activating CD4+CD25+ regulatory T cells for use in the treatment of a rheumatic disease in a patient, wherein the patient is undergoing methotrexate treatment. In a further alternative aspect the present invention provides a composition comprising methotrexate for use in the treatment of a rheumatic disease in a patient, wherein the patient is undergoing treatment with an agent capable of activating CD4+CD25+ regulatory T cells.

In a still further aspect the present invention provides an agent capable of activating CD4+CD25+ regulatory T cells and methotrexate as a combined preparation for simultaneous, separate or sequential use in the treatment of a rheumatic disease in a patient who is a non-responder to treatment with a disease modifying anti-rheumatic drug (DMARD).

### BRIEF DESCRIPTION OF THE DRAWINGS

The invention will be illustrated by way of example only, with reference to the following Figures, in which:

FIG. 1 shows the results of an in vitro proliferation assay conducted with CD4+CD25+ regulatory T cells taken from two donors (Exp. 1 and Exp. 2) in Example 1.

FIGS. 2A and 2B show graphs of the % of patients achieving at least an ACR 20 score over the course of the clinical trial described in Example 2 as compared with patients in the most effective dose groups reported in phase III trials published by Keystone et al., (2004) and (2009). The graph in FIG. 2B is placebo-corrected.

FIGS. 3A and 3B show graphs of the % of patients achieving at least an ACR 50 score over the course of the clinical trial described in Example 2 as compared with patients in the most effective dose groups reported in phase III trials published by Keystone et al., (2004) and (2009). The graph in FIG. 3B is placebo-corrected.

FIGS. 4A and 4B show graphs of the % of patients achieving at least an ACR 70 score over the course of the clinical trial described in Example 2 as compared with patients in the most effective dose groups reported in phase III trials published by Keystone et al., (2004) and (2009). The graph in FIG. 4B is placebo-corrected.

FIG. 5 shows the nucleotide sequence (SEQ ID No: 3) of a fragment of the plasmid encoding the V_{H} region of humanized B-F5. The sequence encoding the V region is underlined and the corresponding polypeptide sequence (SEQ ID No: 15) is indicated below the nucleotide sequence;

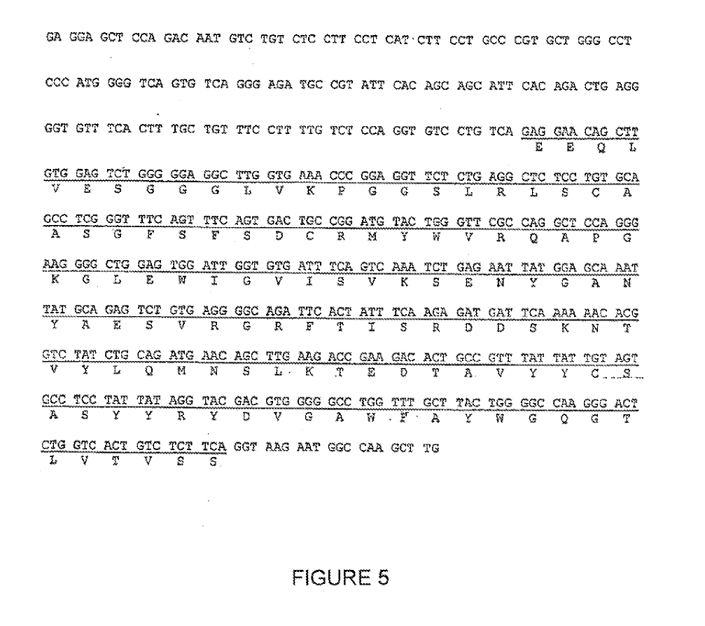

FIG. 6 shows the nucleotide sequence (SEQ ID No: 4) of a fragment of the plasmid encoding the V_{K} regions of humanized B-F5. The sequence encoding the V region is underlined and the corresponding polypeptide sequence (SEQ ID No: 2) is indicated below the nucleotide sequence;

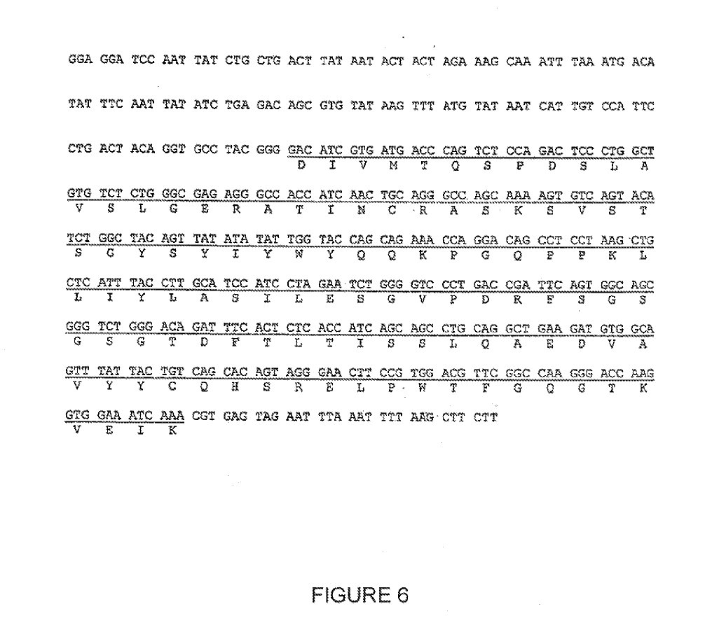

FIG. 7 shows the alignment of the polypeptide sequences of murine B-F5 V_{K} (SEQ ID No: 6), FK-001 (SEQ ID Nos: 7, 8 9 and 10), L4L (SEQ ID No: 16), and L4M (SEQ ID No: 2) in the design of the humanised form of B-F5 (i.e. BT061).

FIG. 8 shows the alignment of the polypeptide sequences of murine B-F5 V_{H} (SEQ ID No: 5), M26 (SEQ ID Nos: 11, 12, 13 and 14), H37L (SEQ ID No: 1), and H37V (SEQ ID No: 15) in the design of the humanised form of B-F5.

### DESCRIPTION OF THE PREFERRED EMBODIMENTS

As described above, the present invention provides a pharmaceutical composition comprising an agent capable of activating CD4+CD25+ regulatory T cells and methotrexate. The present invention further provides a kit comprising separately an agent capable of activating CD4+CD25+ regulatory T cells and methotrexate.

The agent capable of activating CD4+CD25+ regulatory T cells and the methotrexate may be in a single formulation or in separate formulations. The formulations may consist of the agent and/or the methotrexate. Alternatively, the formulations may comprise the agent and/or the methotrexate, and further comprise pharmaceutically acceptable components, such as carriers or excipients.

In one aspect of the invention the agent and/or methotrexate are adapted for parenteral administration, preferably intramuscular, intravenous or subcutaneous administration. It is most preferred that the agent and/or methotrexate are suitable for subcutaneous administration.

In one embodiment of this aspect of the invention the composition, agent and/or methotrexate are adapted for intravenous administration and are provided in a dosage volume of 0.5 to 500 ml or in a form for dilution to the dosage volume of 0.5 to 500 ml. In an alternative embodiment the composition is suitable for subcutaneous or intramuscular administration and is provided in a dosage volume of 0.1 to 3 ml. Alternatively, the composition, agent and/or methotrexate are suitable for providing a dosage volume of 0.5 to 1.5 ml or 15 to 25 ml.

In an alternative aspect the methotrexate is adapted for oral administration and may be in tablet form.

In further aspects of the invention the composition or kit may be suitable for use as a single dose or suitable for use as part of a plurality of doses, in particular, where the dose is to be administered weekly, once every two weeks, once every four weeks, once every six weeks or once every eight weeks.

In one embodiment of this aspect the kit of the invention comprises a plurality of separate doses of the agent and the methotrexate. In a further embodiment, a dosage pack is provided comprising a plurality of separately packaged doses of the pharmaceutical composition.

In one specific embodiment the agent, optionally with the methotrexate, is suitable for subcutaneous administration and is provided in a ready for administration form which does not require dilution so that they can be easily administered by non-medical personnel.

The agents that are suitable for use in the present invention are those which are capable of activating CD4+CD25+ regulatory T cells. The agent may be a polypeptide, a protein, or an antibody or fragment or derivative thereof. Where the agent is an antibody it may be a monoclonal antibody, preferably a humanized monoclonal antibody. The agent may be an anti-CD4 antibody or fragment or derivative thereof. Preferably the antibody is a monoclonal anti-CD4 antibody. The antibody may also preferably be an IgG1 antibody and may be an unmodified IgG1 antibody.

The antibodies which are most suitable for use in the present invention are humanized anti-CD4 antibodies, or fragments or derivatives thereof, which are capable of activating CD4+CD25+ regulatory T cells. Examples of antibodies which are capable of activating CD4+CD25+ regulatory T cells are discussed in Becker et al., (European Journal of Immunology (2007), Vol. 37: pages 1217-1223) and are described in WO 2004/083247.

Generally the antibody used in the invention comprises one or more variable domains which are capable of binding to CD4. The antibody may comprise a human constant region (Fc). This constant region can be selected among constant domains from any class of immunoglobulins, including IgM, IgG, IgD, IgA and IgE, and any isotype, including IgG1, IgG2, IgG3 and IgG4. Preferred constant regions are selected among constant domains of IgG, in particular IgG1.

The present invention also includes any fragment of the antibody. Fragments preferably comprise the antigen binding or V regions of the antibody, and are in particular Fab, Fab′, F(ab)′₂, Fv and scFv fragments.

In a particularly preferred aspect of the present invention the antibody is a humanized anti-CD4 antibody or fragment or derivative thereof derived from the mouse monoclonal anti-CD4 antibody B-F5. The antibody may be a humanized anti-CD4 antibody which comprises a sequence comprising the complementarity-determining regions (CDRs) of the mouse monoclonal antibody B-F5, optionally with variations in the sequence that do not substantially affect the antibody specificity and/or affinity thereof.

Examples of antibodies are provided in WO 2004/083247, in which the production of several humanised versions of the mouse B-F5 antibody is disclosed. In particular, WO 2004/083247 discloses the production of a humanised antibody BT061 (hB-F5) having V domains defined by the following polypeptide sequences:

H Chain V Domain:

(SEQ ID NO: 1)
EEQLVESGGGLVKPGGSLRLSCAASGFSFSDCRMYWLRQAPGKGLEWI
GVISVKSENYGANYAESVRGRFTISRDDSKNTVYLQMNSLKTEDTAVY
YCSAS YYRYDVGAWFAYWGQGTLVTVSS

L Chain V Domain:

(SEQ ID NO: 2)
DIVMTQSPDSLAVSLGERATINCRASKSVSTSGYSYIYWYQQKPGQPP
KLLIYLASILESGVPDRFSGSGSGTDFTLTISSLQAEDVAVYYCQHSR
ELPWTFGQGTKVEIK.

Derivatives of this antibody are also suitable for use in the present invention. Derivatives include those with V domains defined by polypeptide sequences having at least 80%, preferably at least 90%, most preferably at least 95% sequence identity with SEQ ID NO: 1 or SEQ ID NO: 2.

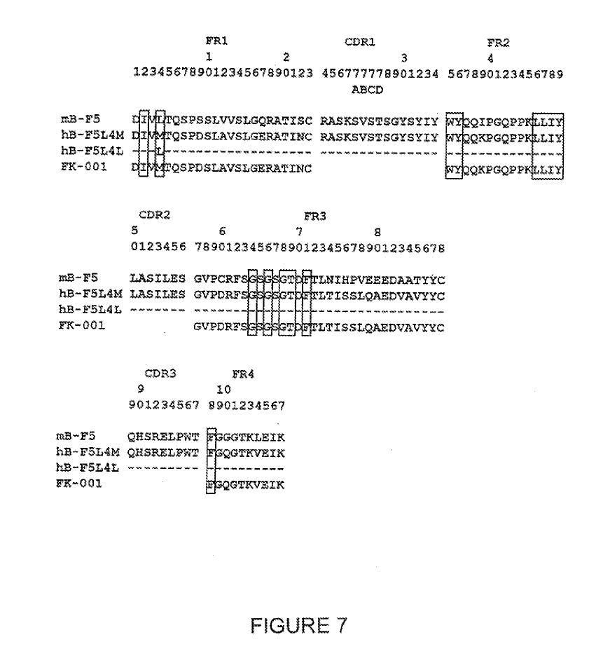

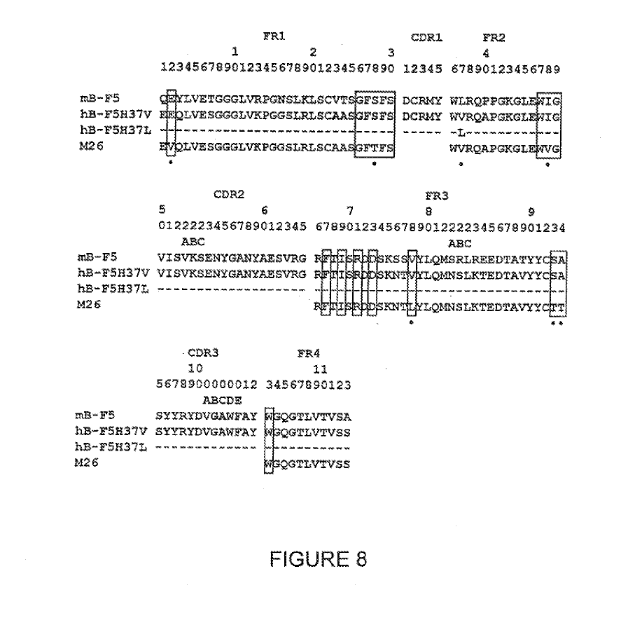

Particularly preferred antibodies are those which comprise the complementarity-determining regions (CDRs) of the mouse B-F5 mAb, and retain the ability of hB-F5 to activate CD4+ CD25+ regulatory T cells. The location of the CDRs within the V_{H} and V_{K} domains is shown in FIGS. 7 and 8. Such antibodies can optionally have variations in the sequence of the CDRs that do not substantially affect the specificity and/or affinity of binding.

Generally, the antibody used in the invention further comprises a human constant region (Fc). This constant region can be selected from among the constant domains indicated above.

The present invention also includes any fragment of the hB-F5 antibody or derivative thereof comprising the V regions thereof. This comprises in particular Fab, Fab′, F(ab)′₂, Fv and scFv fragments.

In order to prepare the hB-F5 antibody a polynucleotide encoding the V domain of the H chain or of the L chain of a BT061 antibody may be fused with a polynucleotide coding for the constant region of a human H or L chain. For the purpose of expressing the complete H and L chains obtained in this way a sequence coding a signal peptide allowing the secretion of the protein can also be added.

The polynucleotide as described above is linked within an expression vector to appropriate control sequences allowing the regulation of its transcription and translation in a chosen host cell. These recombinant DNA constructs can be obtained and introduced in host cells by the well-known techniques of recombinant DNA and genetic engineering.

Useful host-cells can be prokaryotic or eukaryotic cells. Among suitable eukaryotic cells, one will mention, by way of example, plant cells, cells of yeasts such as Saccharomyces, cells of insects such as Drosophila, or Spodoptera, and mammal cells such as HeLa, CHO, 3T3, C127, BHK, COS, etc.

The BT061 (hB-F5) antibody utilised in the invention can be obtained by culturing a host cell containing an expression vector comprising a nucleic acid sequence encoding said antibody, under conditions suitable for the expression thereof, and recovering said antibody from the host cell culture.

A further aspect of the invention is a method comprising preparing a kit or a pharmaceutical composition comprising the agent and the methotrexate.

As indicated above, the present invention further provides medical uses and methods of treatment of patients suffering from, or susceptible to rheumatic diseases. In particular, in one aspect a method of treating a rheumatic disease in a patient is provided comprising a step (a) of administering an agent capable of activating CD4+CD25+ regulatory T cells and a step (b) of administering methotrexate, wherein step (a) and step (b) can be conducted simultaneously, separately or sequentially and in either order. In one embodiment of this aspect step (a) and step (b) are conducted on the same day. In an alternative embodiment of this aspect step (a) and step (b) are conducted within the same week.

In alternative aspects, the present invention provides a method of treating a rheumatic disease in a patient undergoing methotrexate treatment comprising a step of administering an agent capable of activating CD4+CD25+ regulatory T cells, and a method of treating a rheumatic disease in a patient undergoing treatment with an agent capable of activating CD4+CD25+ regulatory T cells comprising a step of administering methotrexate.

Rheumatic diseases are defined as diseases affecting the connective tissue, especially the joints and related structures, in particular being characterized by inflammation, degeneration or metabolic derangement. In a preferred aspect of the invention the rheumatic disease is rheumatoid arthritis, psoriatic arthritis, juvenile rheumatoid arthritis or ankylosing spondylitis.

The treatment of rheumatoid arthritis is preferred. With rheumatoid arthritis clinical efficacy of treatment may be assessed using ACR scoring.

ACR scoring is a method of assessment of rheumatoid arthritis exhibited by a treated patient set out by the American College of Rheumatology (ACR) and works through the measurement of a core set of parameters (Felson et al., Arthritis & Rheumatism, 1995, 38(6), 727-735). This system defines a value of ACR 20 as an at least 20% improvement in tender and swollen joint counts and at least 20% improvement in 3 of the 5 remaining ACR core set measures: patient and physician global assessments, pain, disability, and an acute phase reactant, such as C-reactive protein (CRP) or Erythrocyte Sedimentation Rate (ESR). Similarly, ACR 50 and ACR 70 scores define an at least 50% and an at least 70% improvement, respectively.

In a further aspect of the invention the treatment may be administered to a patient who is a non-responder to treatment with a disease-modifying anti-rheumatic drug (DMARD). A non-responder is a patient who shows an inadequate response to treatment with a DMARD. In particular, a patient shows an inadequate response if he/she has continuing clinically active rheumatoid arthritis, e.g. if the drug is not achieving ACR20 in the patient or is not achieving an inhibition of progression of structural damage to the joints or if an initial response to the drug is lost over time during treatment.

Examples of DMARDs are e.g., hydroxycloroquine, sulfasalazine, methotrexate, penicillinamine, cyclophosphamide, gold salts, azothipoprine, leflunomide, etc.

As indicated above, the agent and the methotrexate may be administered to the patient in any suitable manner. In particular, they may be administered parenterally, for example by intravenous, intramuscular or subcutaneous injection. For administration of the agent, intravenous or subcutaneous administration is particularly preferred. Further, methotrexate may be administered orally.

The volume in which the agent and/or methotrexate are dosed will vary depending on the method of administration. Where the dose is to be administered by intravenous infusion the dosage volume may be from 0.1 or 0.5 ml up to 500 ml, preferably between 15 and 25 ml, and typically about 20 ml. Where the dose is to be administered by subcutaneous or intramuscular injection, the dosage volume may be between 0.1 to 3 ml, preferably between 0.5 and 1.5 ml, and typically about 1 ml.

The frequency of administration is not especially limited, provided that it does not interfere with the effectiveness of the treatment. Treatment may comprise a single dose or a plurality of doses. It is preferred that the plurality of doses are administered on at least the following bases: weekly, every two weeks, every 4 weeks, every 6 weeks, every 12 weeks, every 24 weeks, every calendar month, every 6 calendar months or yearly. Thus, the doses may be separated by at least one week, or by at least two weeks, at least one month or by at least 3 months or by at least 6 months or by at least one year (meaning that the doses are taken every week, every two weeks, or every month or every 6 months or every year). It is particularly preferred that the doses are administered at least every two to three weeks.

The length of treatment is not especially limited, and, as is typical in the treatment of autoimmune diseases, the treatment proceeds indefinitely, or until symptoms are reduced to a manageable level for the patient. Generally the treatment is administered to the subject for at least 1 month.

It will be understood that the agent and the methotrexate are to be administered in therapeutically effective amounts, i.e. in amounts which are effective for ameliorating, preventing or treating the rheumatic disease.

In particular, where the disease is rheumatoid arthritis, the agent and the methotrexate are preferably administered in an amount that is effective to provide an ACR50 response, more preferably an ACR70 response.

In one aspect of the invention the agent is to be administered to a subject in a dose from 0.2 to 10 mg, and more preferably in a dose from 0.2 to 6.25 mg or 0.2 to 5 mg, and most preferably in a dose from 0.2 to 3 mg or 0.5 to 3 mg. These doses are particularly preferred where the dose is administered intravenously.

Where the agent is the humanised antibody BT061 the inventors have surprisingly found that the effective Cmax values of the antibody circulating in the plasma of healthy volunteers 3 hours after the end of intravenous infusion are much lower than expected, as shown in Table 1, below. This is considered to reflect a faster target mediated clearance.

TABLE 1
the expected and effective plasma levels of BT061 after intravenous
administration. The range of plasma levels measured in three different
volunteers per dose-group is indicated.

| | plasma level (μg/ml) | | |
|---|---|---|---|
| | theoretical | effective cmax (post | conc. 3 h post end of |
| dosage (mg) | cmax | end of infusion) | infusion |
| 2.5 | 0.71 | 0.06-0.14 | 0 |
| 5 | 1.43 | 0.32-0.44 | 0.09-0.16 |
| 10 | 2.86 | 0.8-2.0 | 0.46-1.4 |
| 20 | 5.71 | 2.3-4.1 | 1.7-3.3 |
| 40 | 11.43 | 5.9-6.5 | 4.4-6.1 |
| 60 | 17.14 | 8.9-15.1 | 7.7-11.08 |

Accordingly, in a preferred embodiment of the invention 0.2 to 10 mg of the agent is administered intravenously and the maximum concentration of the agent in the patient's plasma three hours after the administration is less than 2.5 μg/ml. Preferably, 0.2 to 5 mg of the agent is administered intravenously and the maximum concentration of the agent in the patient's plasma three hours after the administration is less than 0.3 μg/ml. Still more preferably, 0.5 to 3 mg of the agent is administered intravenously and the maximum concentration of the agent in the patient's plasma three hours after the administration is less than 0.1 μg/ml. These values are obtained after any administration and/or after the first and/or second administration of the agent.

The dose can also be calculated on the basis of the body surface area (BSA) of the subject. Body surface area (BSA) can be calculated according to any known method. Examples of BSA calculation methods are: the Mosteller formula of (BSA (m²)=([Height (cm)×Weight (kg)]/3600)^{1/2} (Mosteller R D., N Engl J Med 1987 Oct. 22; 317(17):1098); the DuBois and DuBois formula of BSA (m²)=0.20247×Height (m)^{0.725}×Weight (kg)^{0.425} (DuBois D; DuBois E F., Arch Int Med 1916 17:863-71); the Haycock formula of BSA (m²)=0.024265×Height (cm)^{0.3964}×Weight (kg)^{0.5378} (Haycock G. B., et al., The Journal of Pediatrics 1978 93:1:62-66); the Gehan and George formula of BSA (m²)=0.0235×Height (cm)^{0.42246}×Weight (kg)^{0.51456} (Gehan E A, and George S L, Cancer Chemother Rep 1970 54:225-35); and the Boyd formula: BSA (m²)=0.0003207×Height (cm)^{0.3}×Weight (grams)^{(0.7285-(0.0188×LOG (grams))}.

Accordingly, the agent can be administered to a subject in a dose from 0.1 to 5 mg/m² body surface area of the patient, preferably from 0.1 to 2.5 mg/m² and most preferably from 0.25 to 1.5 mg/m². Alternatively, the dose can be calculated based on the body weight of the subject, such that in a further aspect of the invention the agent is to be administered to a subject in a dose from 2 to 150 μg/kg, preferably 2 to 75 μg/kg, and most preferably from 5 to 45 μg/kg. As above, it is particularly preferred that these dosages are utilised when the agent is administered intravenously.

As indicated above, in one aspect of the invention the agent and/or methotrexate are administered subcutaneously. In general, as is known in the art, subcutaneous doses need to be larger than intravenous doses in order to achieve the equivalent therapeutic effect. The present inventors have demonstrated in monotherapy trials in rheumatoid arthritis patients with the antibody BT061 that the therapeutic effect achieved after 2 mg intravenous administration is approximately equivalent to that achieved after a 50 mg subcutaneous administration. These results are represented below in Table 2.

TABLE 2
the percentage of rheumatoid arthritis patients showing ACR
improvements at week 7 after being treated with once weekly doses of
BT061 (2 mg intravenous or 50 mg subcutaneous) for a total treatment
period of six weeks. The results represent blinded data from 8 individuals,
2 receiving placebo and six receiving BT061.

| | ACR20 | | |
|---|---|---|---|
| week 7 | (%) | ACR50 (%) | ACR70 (%) |
| 2 mg i.v | 50 | 25 | 12.5 |
| 50 mg s.c. | 50 | 25 | 12.5 |

Accordingly, in a further preferred aspect of the invention the agent is administered to the patient subcutaneously or intramuscularly in a dose from 20 mg to 80 mg, and more preferably from 30 mg to 70 mg. Alternatively, the agent can be administered in a dosage from 8 to 50 mg/m² or from 0.2 to 1.5 mg/kg. In this aspect of the invention it is particularly preferred that the administration is at most about once every two weeks. It is noted that the aspects of the invention described in this paragraph can be combined with the other aspects and preferred features of the invention set out in this application.

According to the present invention the pharmaceutical composition or kit further comprises methotrexate (MTX). MTX treatment of RA is well known in the art and it is envisaged that in the present invention MTX is to be administered in the dosages previously described. In particular, in the invention MTX is usually administered in a dose between 5 to 30 mg, preferably between 7.5 mg and 30 mg and most preferably between 10 to 25 mg. In some cases the dose will depend on the patient's pretreatment with MTX or tolerance to this drug.

In another aspect the method of the invention includes a further step of administering an additional therapeutic agent suitable for treating the rheumatic disease. Additional therapeutic agents comprise one or more of the following agents: a non-steroidal anti-inflammatory drug, an anti-inflammatory steroid, a gold compound, an anti-malarial drug, folic acid, cyclosporine, leflunomide, azathioprine, sulfasalazine, d-penicillamine, cyclophosphamide, mycophenoate, minocycline and chlorambucil. These additional therapeutic agents may be administered separately, simultaneously or sequentially with the agent capable of activating CD4+CD25+ regulatory T cells and the MTX.

As mentioned above the present inventors have surprisingly shown that the pharmaceutical composition and kit of the present invention are capable of treating rheumatic diseases. In particular, as shown below in Example 2 the treatment of rheumatoid arthritis results in a significant improvement in the disease. Accordingly, in a still further aspect of the invention where the disease is rheumatoid arthritis the treatment provides an improvement of the disease in the patient of at least ACR20, preferably at least ACR 50, and more preferably at least ACR 70, according to the American College of Rheumatology scoring system. In other words the treatment provides an at least 20%, preferably at least 50% and most preferably at least 70% improvement of the disease parameters according to the American College of Rheumatology (ACR) score of the patient.

Most preferably the treatment provides at least an ACR 70 response in the patient between 6 to 8 weeks after the start of treatment.

As can also be seen from Example 2 and the related Figures, the treatment of the present invention has the capacity to improve rheumatoid arthritis in a number of patients. Accordingly, the method of treatment of the present invention is capable of treating rheumatoid arthritis by providing at least an improvement of the disease condition of ACR 20 to at least 20% of patients. Further, the method of treatment of the present invention is capable of treating rheumatoid arthritis by providing at least an improvement of the disease condition of ACR 50, more preferably ACR 70, to at least 10% of patients.

The invention will now be described further with the following examples.

### Example 1

### In Vitro Proliferation Assay with the Antibody BT061 Using Freshly Isolated CD4+CD25+ Regulatory T Cells

Method

Isolation of Human CD4+CD25+ Regulatory T Cells

50 ml EDTA blood specimens were obtained from healthy control donors. Peripheral blood mononuclear cells (PBMCs), regulatory T cells (Tregs) and T helper cells as T responder cells (Tresps) were isolated from peripheral blood samples as previously described (Haas et al., 2007).

In Vitro Proliferation Assays

Freshly isolated Tregs were pre-incubated for 48 hours with 1 μg/ml plate bound antibody (BT061), 1 μg/ml soluble BT061 or Medium.

Freshly isolated Tregs (2.5×10⁴, donor A) obtained from 2 donors (Exp. 1 and Exp. 2) were pre-incubated for 48 hours with either 1 μg/ml soluble or plate bound BT061. To achieve allogeneic stimulation the 2.5×10⁴ pre-incubated Tregs were then transferred to 1×10⁵ T cells as responder cells (Tresps) from a second donor (donor B) in the presence of 2×10⁵ T cell depleted and irradiated (30 Gray) PBMCs (donor A). After 4 days of stimulation 1 μCi [3H] thymidine per well was added and proliferation was measured after additional 16 hours.

Results

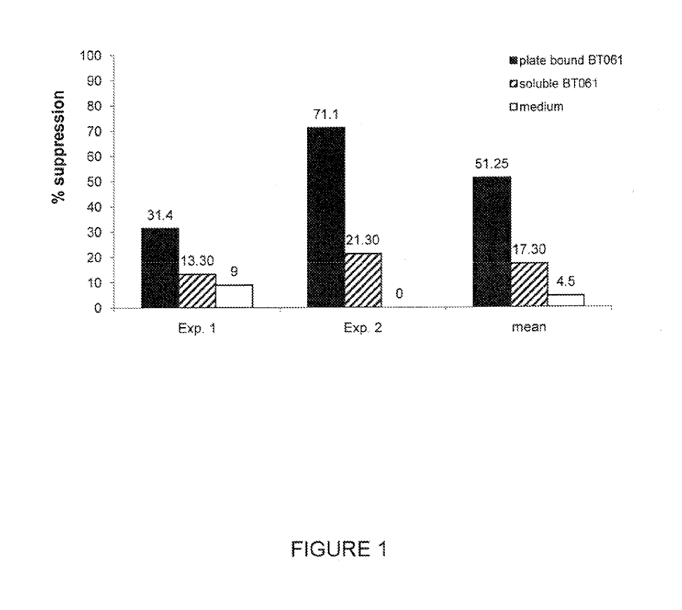

The percentage of Treg mediated inhibition of Tresp proliferation is shown in FIG. 1 as the percent suppression of proliferation of Tresps incubated with PBMC in the absence of Tregs. Results are shown for the Tregs obtained from the two donors (Exp. 1 and Exp. 2). The dashed bars represent the results obtained with the Treg cells pre-incubated with soluble antibody, while the filled bars represent the results obtained with the Treg cells pre-incubated with plate bound antibody. As a control the suppressive activity of medium treated Tregs (open bars) is shown. Numbers above bars represent the percentage inhibition of Tresp proliferation.

As FIG. 1 shows, Tregs pre-incubated with plate bound or soluble antibody were able to reduce average proliferative responses of allogeneic stimulated Tresps in contrast to Tregs incubated with medium alone. Further, the suppression obtained with plate-bound antibody was stronger compared to suppression obtained with soluble antibody.

Under physiological conditions in vivo BT061 as an IgG1 antibody is expected to bind to Fc receptors on Fc receptor expressing cells. This interaction would lead to recruitment of homogenously distributed BT061 (bound to CD4) into the local interaction site of target cells and Fc receptor expressing cells, leading to a cross-linking of BT061 and thus CD4. It is expected that interaction of BT061 with Fc receptor expressing cells confers a similar signal to Treg target cells as observed with the plate-bound antibody as both mechanisms recruit several target molecules (CD4) into close proximity on the cell surface.

### Example 2

### Clinical Trial in Patients with Rheumatoid Arthritis

The ability of the pharmaceutical compositions and kits of the present invention to provide efficacious treatment of RA was demonstrated in patients suffering from RA.

The combination trial in which BT061 was studied in combination with MTX comprised a randomized placebo controlled double blind phase II study conducted in patients with moderate to severe RA. All patients had been taking stable doses of MTX for at least 3 months prior to the start of the trial, and these doses were continued in all patients the range of 15 to 20 mg per week during the course of the trial administered orally or intramuscularly.

The patients were divided into three groups. The patients in group I (14 patients) received a 0.5 mg intravenous dose of BT061 and a dose of MTX in the range of 15 to 20 mg. The patients in group II (42 patients) received a 2.0 mg intravenous dose of BT061 and a dose of MTX in the range of 15 to 20 mg. The patients in group III (14 patients) received a dose of MTX in the range of 15 to 20 mg. The patients were dosed once a week over a period of eight weeks.

For intravenous administration the agent is to be infused in the forearm vein according to medically accepted procedures.

The treatment efficacy was evaluated weekly over the dosing period, and for a number of weeks after dosing was complete, by assessment of the ACR parameters (American Society of Rheumatology ACR homepage) and in particular studying the number of tender and swollen joints and following the levels of C-reactive protein (CRP) and the erythrocyte sedimentation rate (ESR). These parameters were also assessed before the trial to provide a “baseline” value at day 0.

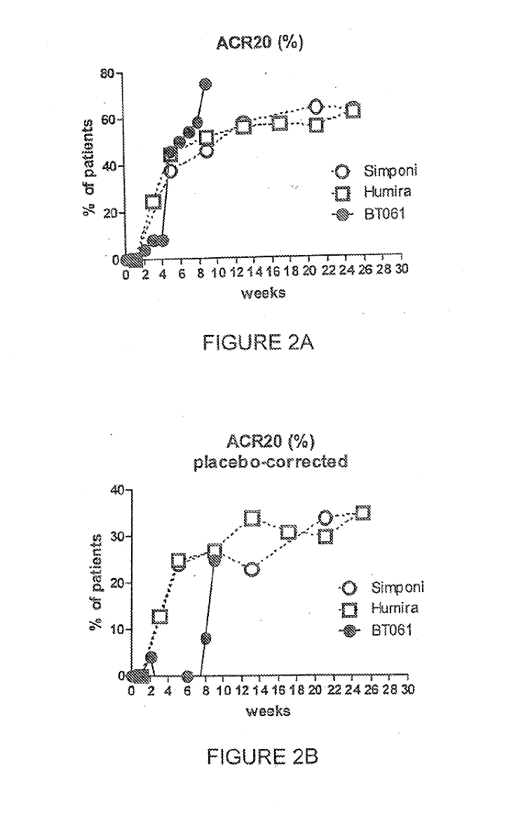

The results of the trial are shown in FIGS. 2 to 4 in which data obtained from patients in dose group II, receiving doses of 2 mg BT061+MTX, are compared with the most effective dose groups obtained in two published phase III trials involving the anti-TNF alpha antibodies, Humira (adalimumab) by Keystone et al., (2004—trial DE019) and Simponi (golimumab) by Keystone et al., (2009—Go-Forward trial) The most effective dose groups are shown for all of the studies.

It should be noted that the prior art results included for comparative purposes are phase III trials in which the doses of the pharmaceutical compositions have been optimized.

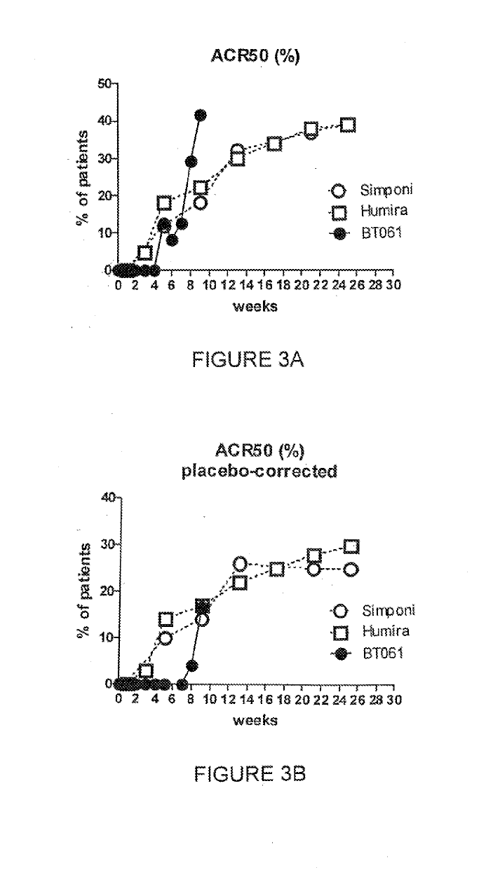

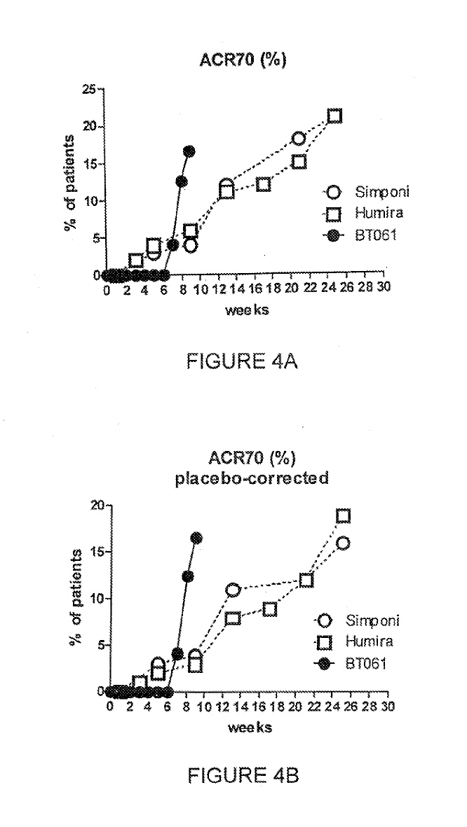

In particular, FIG. 2 shows the percentage of patients from the 2 mg dose group achieving an ACR20 score, while FIGS. 3 and 4 show the percentage of patients from the 2 mg dose group achieving an ACR50 and an ACR 70 score, respectively.

As can be seen in the Figures, in the clinical trials with the therapeutic anti-TNF-alpha antibodies, peak therapeutic activity as measured by improvement of ACR scores usually requires several months. Generally, the percentage of patients showing an ACR20 response is maximal and reaches a plateau after 3 months. For ACR50 the plateau is reached after 4 months and for ACR70 the plateau is reached after 6 months.

However, the results of the combined therapy of the present invention show a number of differences. In particular, in the Figures showing the placebo-corrected results it can be seen that the onset of the therapeutic effect is delayed; the percentage of patients achieving an ACR20 score does not rise above 5% until week 8. However, after onset the therapeutic effect increases rapidly such that at week 9 the percentage of patients achieving ACR50 is comparable to that achieved in the phase III trials with the TNF-alpha antibodies, Humira and Simponi. The percentage of patients reaching ACR 20, ACR 50 and ACR70 increases rapidly between weeks 7 to 9 such that by week 9 the percentage of ACR20, ACR50 and ACR70 patients is approximately 25%, 18% and 17%, respectively. It is noted that this percentage of ACR70 patients is not reached in the trials with Humira and Simponi until 24 to 26 weeks after the start of treatment.

In addition the present inventors have noted that the number of adverse side effects seen with the combination therapy of BT061 and MTX is lower than that seen in the trials performed using BT061 alone, and therefore MTX has the capacity to reduce the side effects of therapeutic antibodies which activate CD4+CD25+ regulatory T cells.

These results demonstrate the efficacy and surprising advantages of the combination of an agent capable of activating CD4+CD25+ regulatory T cells and MTX of the present invention in the treatment of rheumatic disease.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

1. A pharmaceutical composition comprising an agent capable of activating CD4+CD25+ regulatory T cells and methotrexate.

2. A method of treating a rheumatic disease in a patient comprising a step (a) of administering an agent capable of activating CD4+CD25+ regulatory T cells and a step (b) of administering methotrexate, wherein step (a) and step (b) can be conducted simultaneously, separately or sequentially and in either order.

3. A method of treating a rheumatic disease in a patient undergoing methotrexate treatment comprising a step of administering an agent capable of activating CD4+CD25+ regulatory T cells.

4. A method of treating a rheumatic disease in a patient undergoing treatment with an agent capable of activating CD4+CD25+ regulatory T cells comprising a step of administering methotrexate.

5. A method of treating a rheumatic disease in a patient according to claim 2 wherein the rheumatic disease is selected from rheumatoid arthritis, psoriatic arthritis, juvenile rheumatoid arthritis and ankylosing spondylitis.

6. A method of treating a rheumatic disease in a patient according to claim 5, wherein the rheumatic disease is rheumatoid arthritis.

7. A method of treating rheumatoid arthritis in a patient who is a non-responder to treatment with a disease-modifying anti-rheumatic drug (DMARD), comprising a step (a) of administering an agent capable of activating CD4+CD25+ regulatory T cells and a step (b) of administering methotrexate, wherein step (a) and step (b) can be conducted simultaneously, separately or sequentially and in either order.

8. A method of treating rheumatoid arthritis in a patient according to claim 7 wherein the DMARD is methotrexate.

9. A method of treating a rheumatic disease in a patient according to claim 2 wherein the agent is administered parenterally.

10. A method of treating a rheumatic disease in a patient according to claim 9 wherein the agent is administered intramuscularly, intravenously or subcutaneously.

11. A method of treating a rheumatic disease in a patient according to claim 2 wherein the methotrexate is administered orally, intramuscularly, intravenously or subcutaneously.

12. A method of treating a rheumatic disease in a patient according to claim 2 wherein the agent is administered to the patient in an amount from 0.2 to 10 mg.

13. A method of treating a rheumatic disease in a patient according to claim 12 wherein the agent is administered to the patient in an amount from 0.2 to 5 mg.

14. A method of treating a rheumatic disease in a patient according to claim 13 wherein the agent is administered to the patient in an amount from 0.5 to 3 mg.

15. A method of treatment according to claim 12 wherein the agent is administered intravenously and wherein the maximum concentration of the agent in the patient's plasma 3 hours after the end of the administration is less than 2.5 μg/ml.

16. A method of treatment according to claim 13 wherein the agent is administered intravenously and wherein the maximum concentration of the agent in the patient's plasma 3 hours after the end of the administration is less than 0.3 μg/ml.

17. A method of treatment according to claim 14 wherein the agent is administered intravenously and wherein the maximum concentration of the agent in the patient's plasma 3 hours after the end of the administration is less than 0.1 μg/ml.

18. A method of treatment according to claim 12 wherein the agent is administered intravenously once every week and wherein the maximum concentration of the agent in the patient's plasma 3 hours after the end of the second administration is less than 2.5 μg/ml.

19. A method of treatment according to claim 13 wherein the agent is administered intravenously once every week and wherein the maximum concentration of the agent in the patient's plasma 3 hours after the end of the second administration is less than 0.3 μg/ml.

20. A method of treatment according to claim 14 wherein the agent is administered intravenously once every week and wherein the maximum concentration of the agent in the patient's plasma 3 hours after the end of the second administration is less than 0.1 μg/ml.

21. A method of treatment according to claim 12 wherein the agent is administered intravenously once every week and wherein the maximum concentration of the agent in the patient's plasma 3 hours after the end of any administration is less than 2.5 μg/ml.

22. A method of treatment according to claim 13 wherein the agent is administered intravenously once every week and wherein the maximum concentration of the agent in the patient's plasma 3 hours after the end of any administration is less than 0.3 μg/ml.

23. A method of treatment according to claim 14 wherein the agent is administered intravenously once every week and wherein the maximum concentration of the agent in the patient's plasma 3 hours after the end of any administration is less than 0.1 μg/ml.

24. A method of treating a rheumatic disease in a patient according to claim 2 wherein the agent is administered to the patient in an amount from 2 to 150 μg/kg.

25. A method of treating a rheumatic disease in a patient according to claim 24 wherein the agent is administered to the patient in an amount from 2 to 75 μg/kg.

26. A method of treating a rheumatic disease in a patient according to claim 25 wherein the agent is administered to the patient in an amount from 5 to 45 μg/kg.

27. A method of treating a rheumatic disease in a patient according to claim 2 wherein the agent is administered to the patient in an amount from 0.1 to 5 mg/m² body surface area of the patient.

28. A method of treating a rheumatic disease in a patient according to claim 27 wherein the agent is administered to the patient in an amount from 0.1 to 2.5 mg/m² body surface area of the patient.

29. A method of treating a rheumatic disease in a patient according to claim 28 wherein the agent is administered to the patient in an amount from 0.25 to 1.5 mg/m² body surface area of the patient.

30. A method of treating a rheumatic disease according to claim 2 wherein a single dose of the agent is administered to the patient.

31. A method of treating a rheumatic disease according to claim 1 wherein a plurality of doses of the agent are administered to the patient.

32. A method of treating a rheumatic disease according to claim 31 wherein the agent and/or the methotrexate are administered at most every week.

33. A method of treating a rheumatic disease according to claim 32 wherein the agent and/or the methotrexate are administered every two weeks, every three weeks or every four weeks.

34. A method of treating a rheumatic disease according to claim 2 which comprises a further step of administering an additional therapeutic agent suitable for treating the disease, selected from a non-steroidal anti-inflammatory drug, an anti-inflammatory steroid, a gold compound, an anti-malarial drug, folic acid, cyclosporine, leflunomide, azathioprine, sulfasalazine, d-penicillamine, cyclophosphamide, mycophenoate, minocycline and chlorambucil.

35. A method of treating a rheumatic disease according to claim 2 wherein the rheumatic disease is rheumatoid arthritis and wherein the treatment provides an improvement of the disease in the patient of at least ACR50 according to the American College of Rheumatology (ACR) scoring system.

36. A method of treating a rheumatic disease according to claim 35 wherein the treatment provides an improvement of the disease in the patient of at least ACR70 according to the American College of Rheumatology (ACR) scoring system.

37. A method of treating a rheumatic disease according to claim 35 wherein the treatment provides at least an ACR 70 response in the patient between 6 to 8 weeks after the start of treatment.

38. A method according to claim 2 wherein the agent capable of activating CD4+CD25+ regulatory T cells is an antibody or fragment or derivative thereof.

39. A method according to claim 38 wherein the antibody is a humanized monoclonal antibody.

40. A method according to claim 38 wherein the agent is an anti-CD4 antibody or fragment or derivative thereof.

41. A method according to claim 40 wherein the agent is a humanized anti-CD4 antibody or fragment or derivative thereof which comprises a sequence comprising the complementarity-determining regions (CDRs) of the mouse monoclonal antibody B-F5, optionally with variations in the sequence that do not substantially affect the antibody specificity and/or affinity thereof.

42. A method according to claim 40 wherein the agent is a humanized anti-CD4 antibody or fragment or derivative thereof having V domains defined by the following polypeptide sequences:
H chain V domain:
(SEQ ID NO: 1)
EEQLVESGGGLVKPGGSLRLSCAASGFSFSDCRMYWLRQAPGKGLEWI
GVISVKSENYGANYAESVRGRFTISRDDSKNTVYLQMNSLKTEDTAVY
YCSASYYRYDVGAWFAYWGQGTLVTVSS
L chain V domain:
(SEQ ID NO: 2)
DIVMTQSPDSLAVSLGERATINCRASKSVSTSGYSYIYWYQQKPGQPP
KLLIYLASILESGVPDRFSGSGSGTDFTLTISSLQAEDVAVYYCQHSR
ELPWTFGQGTKVEIK.
or V domains comprising polypeptide sequences having at least 80% sequence identity with SEQ ID NO: 1 and SEQ ID NO: 2.

43. A method comprising preparing a pharmaceutical composition comprising the agent and the methotrexate according to claim 1.
